(19) United States
(12) Patent Application Publication
(10) Pub. No.: US 2017/0189228 A1
(43) Pub. Date: Jul. 6, 2017

(54) Real-Time Laser Modulation And Delivery In Ophthalmic Devices For Scanning, Imaging, And Laser Treatment Of The Eye

(71) Applicant: University of Rochester, Rochester, NY (US)

(72) Inventors: Qiang Yang, Rochester, NY (US); Jie Zhang, Pittsford, NY (US)

(21) Appl. No.: 15/313,169

(22) PCT Filed: Jul. 14, 2015
(86) PCT No.: PCT/US15/40396

§ 371 Date: Nov. 22, 2016

Related U.S. Application Data
(60) Provisional application No. 62/024,140, filed on Jul. 14, 2014.

(51) Int. Cl. A61F 9/008; A61B 3/113; G02B 27/10; G02B 26/08; G02B 26/10; G02B 27/00; A61B 3/10; A61B 3/13

(52) U.S. Cl. CPC A61F 9/008; A61B 3/1015; A61B 3/102; A61B 3/113; A61B 3/13; G02B 26/0816; G02B 26/105; G02B 27/0093; G02B 27/106; A61F 2009/00846; A61F 2009/00848; A61F 2009/00851; A61F 2009/00853; A61F 2009/00863

(57) ABSTRACT

Systems and methods of real-time laser control and modulation for ophthalmic devices are described. The systems and methods can be used for precise laser delivery at sub-micron resolution in both laser treatment and scan imaging processes. In various embodiments, the systems can include a laser delivery device useful for surgical eye treatment, a laser delivery device useful for an ophthalmic scan imaging device, or both. In one embodiment, the system includes a laser surgery device integrated with a scan-based ophthalmic imaging apparatus. In such an embodiment, an eye motion signal obtained from the imaging apparatus can be used to provide fine-tuned control of the operation of the surgical laser beam in the laser surgery device.

### CROSS-REFERENCE TO RELATED APPLICATIONS

This application claims priority to PCT international application No. PCT/US15/40396 filed on Jul. 14, 2015, which claims priority to U.S. provisional application No. 62/024,140 filed on Jul. 14, 2014, both of which are incorporated herein by reference in their entireties.

### STATEMENT REGARDING FEDERALLY SPONSORED RESEARCH OR DEVELOPMENT

This invention was made with government support under Grant Nos. EY001319 and EY021166 awarded by National Institutes of Health. The government has certain rights in the invention.

### BACKGROUND

Laser surgery of the eye has been one of the great advances in treating eye diseases because there is no risk of direct infection from the laser light, and lasers are associated with high precision and readily controllable function. Laser surgery has found important applications in treating various eye diseases, such as retinal tears or holes, diabetes retinopathy, macular degeneration, glaucoma, retinal vein occlusions, histoplasmosis, central serous retinopathy, ocular tumors, and post-cataract surgery procedures. However, treatment of a patient's retina at a cellular level can be problematic because areas of the eye not undergoing surgery can be damaged by a surgical laser. Accordingly, laser treatment systems useful for eye treatment applications require that the laser is controlled and stabilized with high precision.

However, currently available laser treatment systems do not generally have the required precision and responsiveness to be used at the cellular level without a stabilizing method. When using such systems, the patient's eyes are usually anesthetized and fixed by means of mechanical suction during treatment. This mechanical stabilization can be uncomfortable and does not provide optimal control of the laser.

Similarly, many types of ophthalmic imaging devices utilize one or more laser beams to scan a subject's eye during the imaging process. These laser beams must be properly controlled or modulated to guarantee smooth and stabilized imaging, and to meet laser power safety protocol. However, currently available ophthalmic imaging systems are subject to the same limitations as surgical laser systems, and therefore also need improved methods of stabilization and control.

Accordingly, there is a continuing need in the art for surgical laser and ophthalmic imaging systems and methods exhibiting stable, high precision control.

### SUMMARY

Systems and methods for treating a subject's eye at any layer from the front surface of the eye ball to the retina are described herein. In one embodiment, the system is an ophthalmic laser surgery system, comprising: an ophthalmic imaging apparatus, a surgical light source, and a steering mirror communicatively coupled with the imaging apparatus, wherein the steering mirror is located in the pupil conjugate plain of a subject's eye, and wherein when the steering mirror directs a laser beam from the surgical light source onto the subject's eye, backscattered light from the subject's eye is received by the imaging apparatus; the imaging apparatus tracks a motion of the subject's eye from the backscattered light; and the imaging apparatus sends a control signal based on the motion of the subject's eye to the steering mirror to direct the location of the laser beam. In one embodiment, the surgical laser or light source can be integrated into the imaging or scanning apparatus.

In another embodiment, the system is an ophthalmic laser surgery system, comprising: an ophthalmic imaging apparatus, a surgical light source, a surgical steering mirror communicatively coupled with the imaging apparatus, or an imaging light source, and an imaging steering mirror communicatively coupled with the imaging apparatus, wherein the surgical steering mirror, imaging steering mirror, and scanners can be located in the pupil conjugate plain of a subject's eye, and wherein when the imaging steering mirror directs a laser beam from the imaging light source onto the subject's eye, backscattered light from the subject's eye is received by the imaging apparatus; the imaging apparatus tracks a motion of the subject's eye from the backscattered light; and the imaging apparatus sends a control signal based on the motion of the subject's eye to the imaging steering mirror to direct the location of the imaging light beam, and to the surgical steering mirror to direct the location of a surgical laser beam from the surgical light source.

In various embodiments, the system can comprise other components. In one embodiment, the system further comprises a wavefront sensor for detecting an aberration in the subject's eye. In one embodiment, the system further comprises a dichroic mirror for directing a portion of the backscattered light to the wavefront sensor. In one embodiment, the system further comprises a beam splitter for splitting the beam of backscattered light, wherein a portion of the backscattered light is sent to the imaging apparatus and a portion of the backscattered light is sent to the wavefront sensor. In one embodiment, the system further comprises a stabilization/wavefront corrector communicatively coupled with the imaging apparatus and wavefront sensor. In one embodiment, the stabilization/wavefront corrector sends a control signal to the steering mirror based on the motion and aberration of the subject's eye. In another embodiment, the stabilization/wavefront corrector sends a control signal to the imaging steering mirror and surgical steering mirror based on the motion and aberration of the subject's eye.

In various embodiments of the system, the imaging apparatus is selected from the group consisting of: ocular coherence tomography (OCT) device, scanning laser ophthalmoscope (SLO), adaptive optics scanning light ophthalmoscope (AOSLO), fundus camera, line scan camera, pupil camera, or adaptive optics flood illumination camera. In various embodiments of the system, the surgical light source is a continuous wave (CW) laser, a pulsed laser, a superluminescent diode (SLD), or any other type of light source. In one embodiment, the system further comprises a laser modulator. In various embodiments, the laser modulator is selected from the group consisting of: direct laser diode modulator, mechano-optical isolator; acousto-optic modulator; electro-optic modulator; magneto-optical modulator; and optical isolator.

In one embodiment, the method is a method for controlling the delivery of an ophthalmic laser, comprising: providing an ophthalmic scan imaging apparatus and one or more ophthalmic light sources, wherein each light source is associated with a steering mirror, and the imaging apparatus is communicatively coupled to the one or more steering mirrors,

imaging a subject's eye with the imaging apparatus to detect one or more parameters of the subject's eye, and adjusting the position of the one or more steering mirrors substantially simultaneously with the detection of the one or more parameters, thereby repositioning the delivery location on the subject's eye of the one or more light beams from the one or more light sources. In one embodiment, the method further comprises the step of modulating the one or more light beams based on the one or more parameters detected.

In one embodiment, the parameter is a motion of the subject's eye. In another embodiment, the parameter is a feature on the subject's retina. In one embodiment, at least one of the ophthalmic light sources is a surgical laser. In various embodiments, the surgical laser is a CW laser, a pulsed laser, or a SLD or other light delivery devices. In one embodiment, the imaging apparatus comprises a wide field of view SLO and a small field of view apparatus.

In embodiments of the system and method having both a wide field of view SLO and a small field of view SLO, the direction of the wide field of view SLO fast-scanning axis is perpendicular to the small field of view apparatus fast-scanning axis, and the wide field of view SLO slow-scanning axis is perpendicular to the small field of view apparatus slow-scanning axis.

### BRIEF DESCRIPTION OF THE DRAWINGS

The following detailed description of embodiments will be better understood when read in conjunction with the appended drawings. It should be understood, however, that the embodiments are not limited to the precise arrangements and instrumentalities shown in the drawings.

FIG. 1 is a schematic diagram of an exemplary embodiment of a laser treatment system.

FIG. 2 is a schematic diagram of another exemplary embodiment of a laser treatment system.

FIG. 3 is a set of images showing an example of image registration failure in images from an AOSLO.

FIG. 4, comprising FIGS. 4A and 4B, is a set of schematic diagrams of exemplary embodiments of an eye tracking system.

FIG. 5 is a schematic diagram of another exemplary embodiment of an eye tracking system.

FIG. 6 is a graph showing data for fixational eye motion in a patient with the disease of cone-rod dystrophy.

FIG. 7 is a graph of image motion data corresponding to images of an eye of a patient with cone-rod dystrophy that was tracked with an embodiment of a tracking system.

FIG. 8 is a single frame of a retinal image from a wide FOV SLO from the eye of a patient with cone-rod dystrophy.

FIG. 9 is a single frame of a retinal image from a wide FOV SLO from the eye of a patient with cone-rod dystrophy showing the size of an exemplary target image (marked with h) and the reference image (marked with II).

FIG. 10 is a set of retinal images from the eye of a patient with cone-rod dystrophy. FIG. 10A is a reference image, featuring strips used for cross-correlation during eye tracking. FIG. 10B is a target image, also featuring strips used for cross-correlation during eye tracking.

FIG. 11 is a schematic diagram of an exemplary embodiment of an electronics system for a wide FOV imaging system.

FIG. 12 is a drawing representing the wide (circle) and small (any square defined by 4 small squares) field of view for an exemplary process of real-time retinal montaging.

FIG. 13, comprising FIGS. 13A and 13B, is a flow chart representing an exemplary eye tracking algorithm.

### DETAILED DESCRIPTION

It is to be understood that the figures and descriptions have been simplified to illustrate elements that are relevant for clear understanding, while eliminating, for the purpose of clarity, many other elements found in the field of image-based eye tracking and scanning-based imaging systems. Those of ordinary skill in the art may recognize that other elements and/or steps are desirable and/or required in implementing the systems and methods described herein. However, because such elements and steps are well known in the art, and because they do not facilitate a better understanding, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art. Any methods and materials similar or equivalent to those described herein can be used in the practice for testing of the systems and methods described herein. In describing and claiming the systems and methods, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. Preferably, the patient, subject or individual is a mammal, and more preferably, a human.

Ranges: throughout this disclosure, various aspects can be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Described herein are systems and methods of real-time laser control and modulation for ophthalmic devices at sub-micron resolution. The systems and methods can be used for precise laser delivery in both laser treatment and scan imaging processes. In one embodiment, the system includes a laser delivery device useful for surgical eye treatment. In another embodiment, the system includes a laser delivery device for an ophthalmic scan imaging device. In another embodiment, the system includes a laser delivery device for both laser surgery and ophthalmic scan imaging. In yet another embodiment, the system includes a laser delivery device for an ophthalmic imaging device. In one embodiment, methods for observing and treating a subject's eye in real-time without the need for a mechanical eye stabilizer and/or anesthetizing the subject's eye are described. In certain embodiments, the systems and methods disclosed herein can include the use of an eye tracking system and method, such as those described in U.S. provisional application No. 62/024,144 filed on Jul. 14, 2014 titled, “System and Method for Real-Time Eye Tracking for a Scanning Laser Ophthalmoscope”, incorporated herein by reference.

In one embodiment, the system includes a laser surgery device integrated with an ophthalmic imaging apparatus. In such an embodiment, an eye motion signal obtained from the imaging apparatus can be used to provide fine-tuned control of the operation of the surgical laser beam in the laser surgery device. The ophthalmic imaging apparatus can include any one of the following devices: ocular coherence tomography (OCT), scanning laser ophthalmoscope (SLO), adaptive optics scanning light ophthalmoscope (AOSLO), fundus camera, line scan camera, pupil camera, or adaptive optics flood illumination camera. In one embodiment, the ophthalmic imaging apparatus of the system can provide a real-time eye motion signal for the purpose of image stabilization during tracking of the eye, and also for precision laser delivery of the surgical laser beam. The method of real-time eye tracking and image stabilization using the imaging apparatus is described in detail later herein.

In various embodiments, the laser surgery beam can be controlled by a pure optical mechanism, such as a steering mirror, galvo mirror, or any other optical mechanism known in the art. In one embodiment, the laser surgery beam can be directed and controlled by manipulating a steering mirror in response to the real-time eye motion data gathered by the imaging apparatus. In such an embodiment, a two-dimensional fast-steering mirror, or two one-dimensional fast-steering mirrors, can be placed at the pupil conjugate to provide a mechanism for quickly and accurately manipulating the surgical laser. A motion signal obtained from the imaging apparatus can be used to create a driving signal, which is then sent to a driver of the one or more steering mirrors, thus changing the laser beam focusing position on the retina in real time.

In one embodiment, the system includes a single laser that serves as the surgical laser which will also scan over the FOV of the imaging system. In such an embodiment, the laser surgery optical system can be integrated with an ophthalmic imaging apparatus through the use of a dichroic mirror. In another embodiment, the system includes two or more separate lasers, i.e., a surgical laser, an imaging laser, and potentially additional lasers. In such an embodiment, the surgical laser and the imaging laser can be separately controlled in response to a real-time motion signal from the imaging apparatus. The one or more laser beams in the system can be manipulated by any optical mechanism known in the art, for example, but not limited to: a 2-D tip-tilt optical mirror, a deformable mirror, or a combination of optical mechanisms.

In various embodiments, the imaging laser beam, the surgical laser beam, or the combined imaging/surgical laser beam of the system can be modulated. Laser modulation can be performed for purposes such as, but not limited to: imaging stabilization; laser delivery accuracy; laser delivery intensity; laser power safety; and emergency system shut down. The laser modulation used in the system and methods can include a variety of methods, including, but not limited to: direct laser diode modulation, mechano-optical isolation, i.e., a mechanical shutter; acousto-optic modulation; electro-optic modulation; magneto-optical modulation; and optical isolation. Among these modulation methods, the optical isolator has the best extinction ratio and throughput, and is generally the most cost-effective. In one embodiment, a mechanical isolator can be used for an emergency system shut down and laser safety protocol. In one embodiment, more than one modulation mechanism can be integrated into the system to optimize the control of the one or more lasers.

In one embodiment, the laser beam used for laser surgery in the system can be used as a light source for the imaging apparatus. In such an embodiment, the surgical laser beam can be used to scan the retina, or it can bypass the scanners in the imaging devices and directly illuminate the surgical location. As described herein, the laser delivery location can be accurately controlled and stabilized in sub-micron level using the real-time motion signal captured from the imaging device. Further, the intensity, position, and pattern of the surgical laser can be modulated accordingly depending on whether the laser is being used solely for imaging or whether the laser is being used for surgical treatment in addition to acting as an illumination source.

In one embodiment, the systems and methods can include a mechanism for wavefront correction for aberration-free performance. In such an embodiment, a wavefront sensing system can be used to detect eye aberration and real-time wavefront correction can be applied, for example via a deformable mirror or liquid crystal optical modulator. Focusing of the surgical laser can be adjusted by the wavefront corrector.

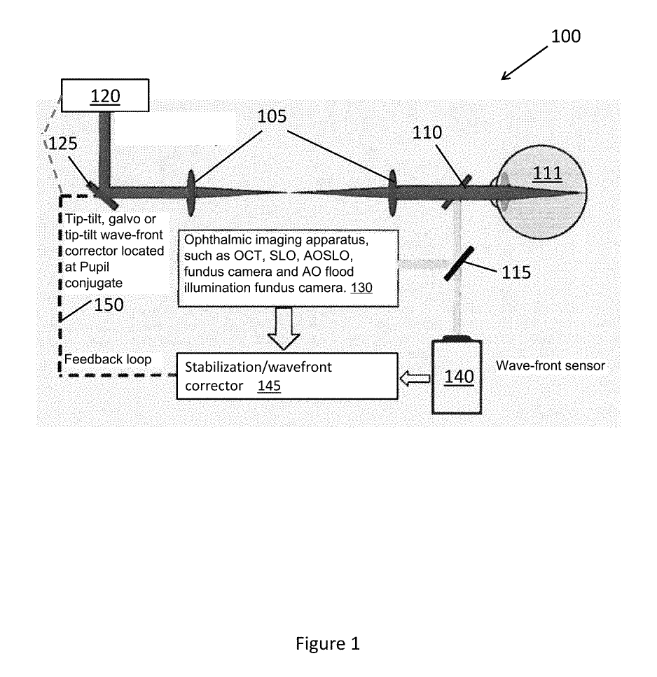

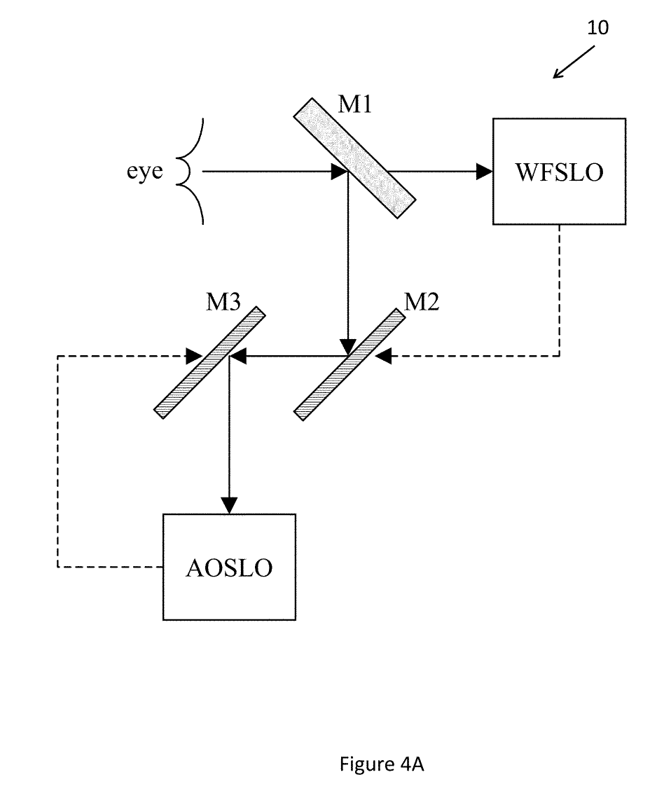

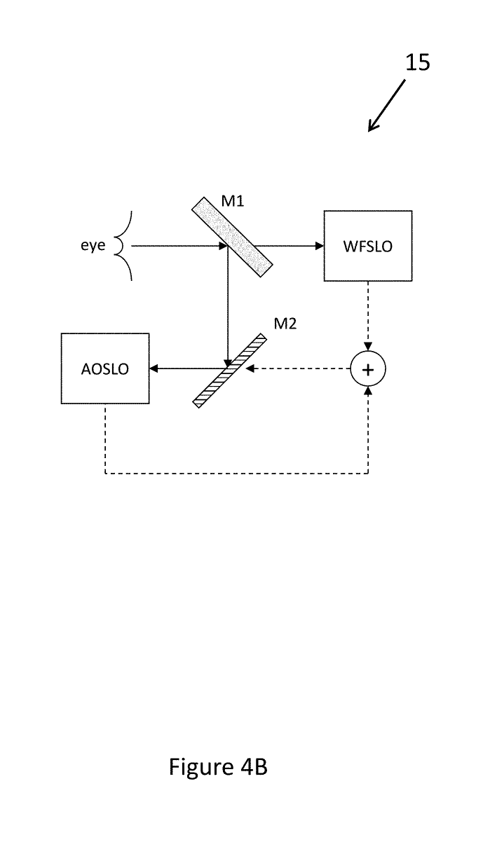

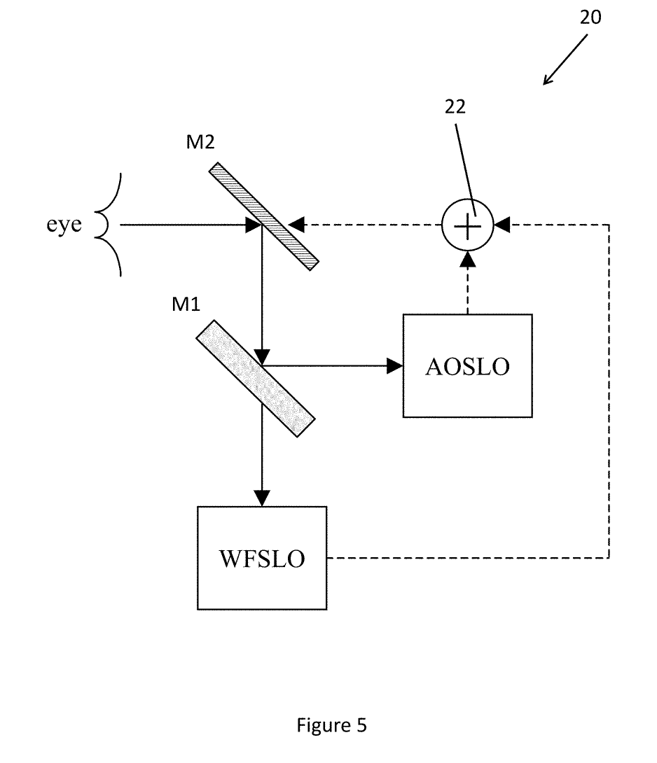

Referring now to FIG. 1, a schematic diagram of an exemplary embodiment of a laser treatment system is shown. System 100 is an imaging-based system which is able to provide precise laser delivery in sub-micron accuracy in real time. System 100 includes a laser source 120 that can produce a laser beam, for example an argon/krypton laser, suitable for laser surgery at various layers across the entire eyeball structure by dynamically focusing the surgical beam and the imaging beams. An ophthalmic imaging apparatus 130 is used to observe and track the motion of the eye 111. Imaging apparatus 130 can be an OCT, SLO, AOSLO, fundus camera, AO flood illumination fundus camera, pupil camera, or any other type of ophthalmic imaging apparatus known in the art. In a preferred embodiment, imaging apparatus 130 is an imaging apparatus as shown in FIG. 4 or 5, which are described in detail later herein. In the embodiment shown in FIG. 1, a single laser beam from laser source 120 acts as both a surgical laser beam and an imaging laser beam, i.e., an imaging illumination source. System 100 also includes a wavefront sensor 140 and stabilization/wavefront corrector 145. System 100 also includes one or more lenses 105, one or more dichroic mirrors or beam splitters 110 and 115, and one or more steering mirrors 125.

In one embodiment, system 100 generally operates as follows. A laser beam is emitted from laser source 120 and reflected by mirror 125 through lenses 105, through dichroic mirror 110, and onto the retina of eye 111. Backscattered light from eye 111 returns to dichroic mirror 110, wherein at least a portion of the backscattered light is reflected onto beam splitter 115. Beam splitter 115 splits the backscattered light into two beams, one of which is incident on an optional wavefront sensor 140, and the other of which is incident on ophthalmic imaging apparatus 130. Accordingly, any motion of eye 111 can be monitored and processed by ophthalmic imaging apparatus 130 to produce a motion signal. The motion signal is then sent to a processor, for example stabilization/wavefront corrector 145, which outputs one or more signals to control the position of the laser from laser source 120 using mirror 125, to control the laser focus using a wavefront corrector, or to control a dedicated auto-focusing system. Wavefront corrector 145 can also receive a wavefront signal from wavefront sensor 140. A processor or other circuitry in wavefront corrector 145 can use the motion signal and the wavefront signal to generate a control signal. The control signal is sent to mirror 125, laser source 120, or both, via a feedback loop 150 to control and/or modulate the real-time position, pattern, and intensity of a laser beam from laser source 120 during laser surgery or eye imaging. Accordingly, mirror 125 can include any necessary components to generate fast and precise motion of the mirror in response to the control signal, such as a driver/motor. Feedback loop 150 can include one or more wired or wireless connections for sending a signal from wavefront corrector 145 to mirror 125 and/or laser source 120.

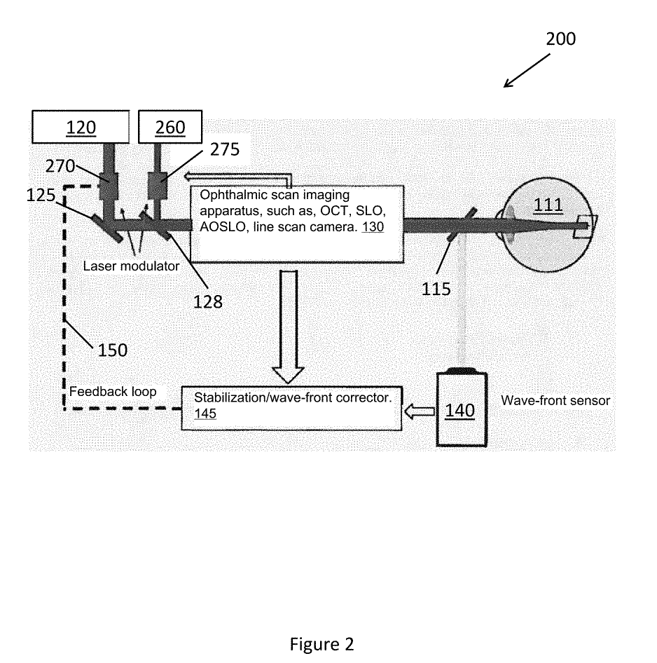

Referring now to FIG. 2, a schematic diagram of another exemplary embodiment of a laser treatment system is shown. System 200 is a system for laser surgery integrated with an ophthalmic scan imaging device and a wavefront sensing system. In this embodiment, system 200 includes a surgery laser source 120 and an imagining laser source 260. In one embodiment, surgery laser source 120 is a significantly more highly powered laser than imaging laser source 260. System 200 includes an ophthalmic scan imaging apparatus 130, an optional wavefront sensor 140, and a stabilization/wavefront corrector 145. System 200 also includes one or more dichroic mirrors 115, one or more steering mirrors 125 and 128, and one or more laser modulators 270 and 275.

In one embodiment, system 200 generally operates as follows. A surgery laser beam and an imaging laser beam can be simultaneously emitted from a surgery laser source 120 and an imaging laser source 260, respectively. Each laser beam is reflected by a separate steering mirror in the pupil conjugate plane, i.e., steering mirrors 125 and 128, onto and through a dichroic mirror 115 and into eye 111. In one embodiment, both the imaging light and surgical laser beams first pass through imaging apparatus 130 prior to passing through dichroic mirror 115. Backscattered light from eye 111 returns to dichroic mirror 115, wherein at least a portion of the backscattered light is reflected onto wavefront sensor 140, and at least a portion of backscattered light is incident on imaging apparatus 130. Accordingly, any motion of eye 111 can be monitored and processed by ophthalmic imaging apparatus 130 and wavefront sensor 140 to produce a motion signal, a wavefront signal, and a focusing signal. These signals are sent to stabilization/wavefront corrector 145 to produce a position and focus control signal for surgical laser source 120. The control signal from wavefront corrector 145 can be sent to steering mirror 125 and/or laser modulator 270 via feedback loop 150 to control or modulate the surgical laser beam being emitted from surgical laser source 120. In addition, a control signal for imaging laser source 260 can be produced by imaging apparatus 130 and sent to steering mirror 128 and/or laser modulator 275 to control or modulate the imaging laser beam being emitted from imaging laser source 260. Accordingly, imaging apparatus 130 and stabilization/wavefront corrector 145 can include any necessary components for creating a control signal, e.g., a microprocessor. Further, steering mirrors 125 and 128 and laser modulators 270 and 275 can include any necessary components for receiving a control signal and implementing a control signal in the mirrors or modulators. It is contemplated herein that a person skilled in the art would understand that components currently available in the art can readily be used to implement the control scheme described herein.

In one embodiment, methods for controlling the delivery of an ophthalmic laser are described herein. The method includes providing an ophthalmic scan imaging apparatus and one or more ophthalmic laser sources, wherein each laser source is associated with a steering mirror, and the imaging apparatus is communicatively coupled to the one or more steering mirrors. The subject's eye is then scanned with the imaging apparatus to detect one or more parameters of the subject's eye, for example any motion of the subject's eye, a feature on the subject's eye that requires surgery with a laser, or any other characteristic or feature of interest of the subject's eye or retina. The one or more laser beams emitted from the ophthalmic laser sources can then be repositioned substantially simultaneously, i.e., in real time, using the steering mirrors based on the information obtained by the imaging apparatus. In one embodiment, the imaging apparatus can also be coupled with one or more laser modulators, wherein the laser beams can be modulated based on the information obtained by the imaging apparatus.

The systems and methods can be used for the treatment of a subject's eye at any layer of the eyeball structure, including, but not limited to the cornea, lens, and retina. The eye surgery techniques can include, but are not limit to, LASIK, Femtosecond Laser Intrastromal Vision Correction, photorefractive keratectomy and laser thermal keratoplasty, phototherapeutic keratectomy, repairing retinal tears or holes, diabetes retinopathy, macular degeneration, glaucoma, retinal vein occlusions, histoplasmosis, central serous retinopathy, ocular tumors and post-surgery cataract procedures.

### Real-Time Eye Tracking Using an Imaging Apparatus

Described herein are systems and methods for real-time eye tracking using a single wide-FOV SLO, a single small-FOV SLO, a combination of both, or some other type of scanning or flood imaging system. The imaging apparatus and methods provide robust and accurate image-based eye tracking for both small and large field SLO, with or without adaptive optics. The imaging apparatus and methods of the present invention are particularly useful for performing eye tracking, i.e., tracking of eye motion from an AOSLO or other imaging system. Also described herein is a method for rapidly re-locking the tracking of a subject's eye position after a microsaccade, a blink, or some other type of interference with image tracking.

Eye tracking requires image registration, which involves relating and aligning the features in a target image with the corresponding features in a reference image. Image registration can be performed “off-line,” wherein a series of high resolution target images are made and then later registered to the reference image. Image registration can also be performed in real-time, wherein features on target images are continuously mapped or registered to the reference image as each target image is being produced. Accurate real-time image registration in ophthalmoscopy is significantly more difficult than off-line registration for a number of reasons. For example, eye motion in the subject can interfere with or prevent accurate image tracking. Further, the light-absorbing nature of a subject's retina generally results in images of the retina having low resolution features. The low resolution of these features make them difficult to track and can result in artifacts being confused with features of the subject's eye.

Two types of systems can be used for eye tracking in ophthalmoscopy: a wide FOV system such as a SLO, a line-scan system, or a flood imaging system, operating within a range on the order of tens of degrees, and a small FOV system such as an AOSLO, operating within about 1 to 2 degrees. The example below includes the combination of a wide-FOV SLO and a small-FOV AOSLO.

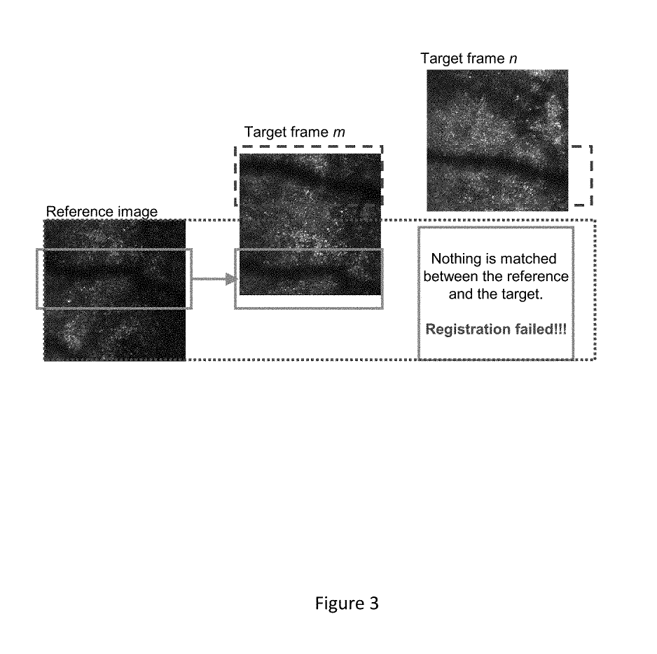

In an image-based eye tracking system where the motion of the eye comes from images from the imaging devices, a wide FOV SLO is capable of covering large eye motion, but it generally does not have high spatial resolution. An AOSLO has high spatial resolution, but frequently suffers from “frame out,” where the target images move out of the reference frame and cause failure of the tracking algorithm. For example, FIG. 3 illustrates the difficulty in maintaining image position in the reference location when using a small FOV system without eye tracking. FIG. 3 is a set of example images from an AOSLO system with a 1.5°×1.5° FOV. In this example, the movement of the target image out of the mapping area of the reference frame was caused by constant eye motion in the subject. Accordingly, it is difficult to effectively implement either wide or small FOV SLO alone in real-time in high resolution because a wide FOV SLO has insufficient spatial resolution and a small FOV SLO suffers from consistent failure of image registration.

In one embodiment, the imaging apparatus combines a wide FOV SLO and an AOSLO into a hybrid tracking system that includes at least one tracking mirror to compensate for large eye motion on the AOSLO. In this embodiment, a signal corresponding to large eye motion is obtained from the wide FOV system, which has low resolution. After correction is applied via the one or more tracking mirrors, the residual image motion on the small FOV system (AOSLO) is reduced to about 20-50 micrometers, which can be efficiently and quickly captured from an AOSLO by using a fast GPU-based algorithm. Thus, the imaging apparatus can significantly improve the performance and accuracy of functional imaging, e.g., for use in laser surgery system.

One embodiment of the imaging apparatus is shown in FIG. 4A. This system is based on a multi-scale method that can be used to optically stabilize the imaging FOV of a small FOV SLO, for example to compensate for image motion caused by eye motion in the subject being examined. In one embodiment, the small FOV SLO is an AOSLO. In another embodiment, the small FOV SLO does not include adaptive optics. In yet another embodiment, a type of high resolution imaging system other than a SLO can be controlled, for example an optical coherence tomography (OCT) system. The optical system 10 includes a beam splitter M1, a tracking mirror M2, and a second tracking mirror M3. System 10 also includes a wide FOV SLO (WFSLO) and an AOSLO. In this embodiment, tracking mirror M2 is controlled by the WFSLO, and the second tracking mirror M3 is controlled by the AOSLO. Accordingly, tracking mirror M2 is used for coarse-scaled tuning to compensate for large eye motion via a motion signal sent from the WFSLO, while the second tracking mirror M3 is used for fine-tuned image motion via a motion signal sent from the AOSLO. In this embodiment, both M2 and M3 are able to separately compensate for an eye motion of ±3°. Therefore, M2 and M3 in combination can compensate up to ±6° eye motion, which is sufficient for most fixational eye movements, even in eyes with poor fixation.

Eye motion can be defined as R(t), which is a function of time t. In the system shown in FIG. 4, the WFSLO will detect any eye motion R(t) of the subject's eye within the wide FOV. A tracking algorithm is used to determine the amount, if any, of motion that must be applied to mirror M2 to compensate for the detected eye motion R(t). The WFSLO then sends a signal to the tracking mirror M2 to cause a compensation motion in M2 based on R(t). The motion of M2 can be defined as A(t). Therefore, the residual image motion appearing on M3 will be,

R(t)−A(t) (1)

In the loop of M1-WFSLO-M2, the tracking mirror M2 is working in an open loop because the WFSLO controls the motion of M2, but does not detect the effects of any motion of M2. At the same time, the second tracking mirror M3 works in a closed loop with the AOSLO because the AOSLO detects residual image motion by dynamically adjusting M3 to compensate for the residual motion R(t)−A(t) caused by M2. If the motion of M3 is defined as B(t), the residual image motion on the AOSLO will be the amount of,

R(t)−A(t)−B(t) (2)

which is detected by an AOSLO tracking algorithm.

Another embodiment of an eye tracking control system and imaging apparatus is shown in FIG. 4B, which is a simplified version of the system in FIG. 4A. Equations (1) and (2) retain the same format for FIG. 4B. System 15 has a single tracking mirror M2 employed to receive control signals from both the small FOV, i.e., the AOSLO, and the WFSLO via a controller 22. System 15 also includes a beam splitter M1. In this embodiment, the combination of AOSLO closed-loop tracking and WFSLO open-loop tracking can be implemented with a single tracking mirror M2, provided that M2 has sufficient dynamic range to compensate for eye motion.

Another embodiment of an eye tracking control system and imaging apparatus is shown in FIG. 5. System 20 is a simplified tracking control system, where a single tracking mirror M2 is employed to receive control signals from both the small FOV, i.e., the AOSLO, and the WFSLO via a controller 22. System 20 also includes a beam splitter M1. Although the configuration of system 20 eliminates the need for a second tracking or steering mirror, with potentially substantial improvement of tracking performance, system 20 requires significantly higher quality optical components in comparison to system 10 in order to maintain a similar quality of image tracking.

In another embodiment, a system having either a WFSLO only or an AOSLO only can be used. In the case of a system with WFSLO tracking only, the spatial resolution of tracking is relatively low, but the cost of the tracking system is reduced significantly by removing the expensive AOSLO unit. In the case of a system with only AOSLO tracking, high spatial resolution can be achieved, but with frequent tracking failure due to large eye motion, blinks, and microsaccades.

The systems and methods can comprise a safety interlock for the surgical laser. For example, in various embodiments of the system, e.g., a small FOV imaging system only; a wide FOV imaging system only; or a combination of a small FOV system and a wide FOV system, the tracking algorithm can be programmed to turn off the surgical laser immediately in response to a failure of the tracking algorithm, such as whenever a microsaccade or a blink occurs.

The embodiments of the imaging apparatus shown in FIGS. 4A, 4B, and 5 can include other components necessary for operation. It is contemplated herein that a person skilled in the art would readily understand, and be able to identify, such components as standard components known in the art. For example, the tracking and steering mirrors described herein would require a mechanism for moving the mirrors based on a control signal. Such a mechanism could include a component for receiving a control signal, and a motor or driver for moving the mirror.

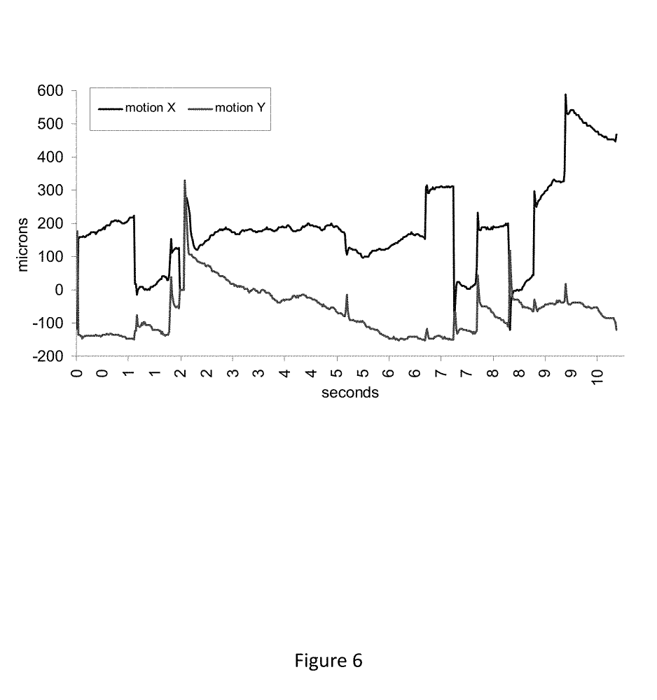

In another aspect, the system and method can distinguish true eye motion signals from artifacts present in the target images. Referring to FIG. 6, a graph representing typical fixational eye motion in a patient having the disease of cone-rod dystrophy is shown. The curves in FIG. 6 have a number of spikes. Some of these spikes correspond to true eye motion signals caused by microsaccades. However, some of the spikes correspond to motion artifacts caused by the low contrast of the captured images, i.e., the spikes correspond to false eye motion signals. When these motion artifacts are treated by the system as actual tracking signals, the tracking mirror is moved unnecessarily, i.e., it jitters, which results in tracking failure. In the imaging apparatus and method, the tracking mirror is generally not moved in relation to these motion artifacts, i.e., the position of the mirror is held constant when the system identifies a motion as a false eye motion signal. Therefore, one element of the imaging apparatus and method is a robust tracking algorithm that is used to distinguish true eye motions from artifacts.

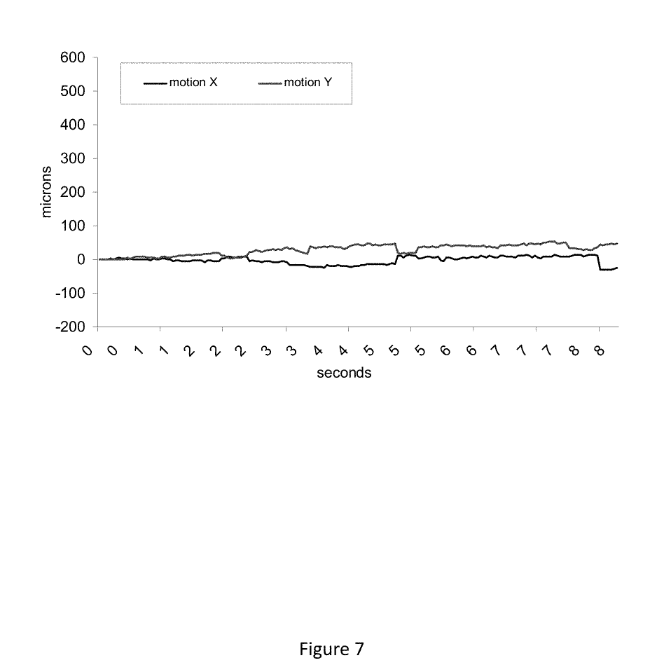

Accordingly, the ability to distinguish true eye motion from false eye motion increases the efficiency and accuracy of the imaging apparatus, which allows for a level of quality in real-time eye tracking unattainable with currently available systems. An example of the reduction in image motion when using the system is shown in FIG. 7. FIG. 7 is a graph showing tracked image motion in a diseased eye where an embodiment of the imaging apparatus employing WFSLO tracking is used for eye tracking. In this particular case, the image motion after the WFSLO tracking is about 1/15 (motion X) and 1/9 (motion Y) of the image motion without WFSLO tracking, as shown in FIG. 6.

Experiments with 20 subjects, 10 having normal eyes and 10 having diseased eyes, showed that tracking performance, in the form of residual image motion, in the direction of fast scan (i.e., motion X in the example) is significantly better than that from the direction of slow scan (i.e., motion Y). Therefore, in optical implementation, WFSLO fast/slow scanning should be perpendicular (rotated 90°) to AOSLO fast/slow scanning, i.e., the WFSLO fast axis should be perpendicular to the AOSLO fast axis, and the WFSLO slow axis should be perpendicular to the AOSLO slow axis. For example, if the WFSLO has fast/slow scanning in X/Y directions, then the AOSLO has fast/slow scanning in Y/X directions. If the WFSLO has fast/slow scanning in Y/X directions, then the AOSLO has fast/slow scanning in X/Y directions.

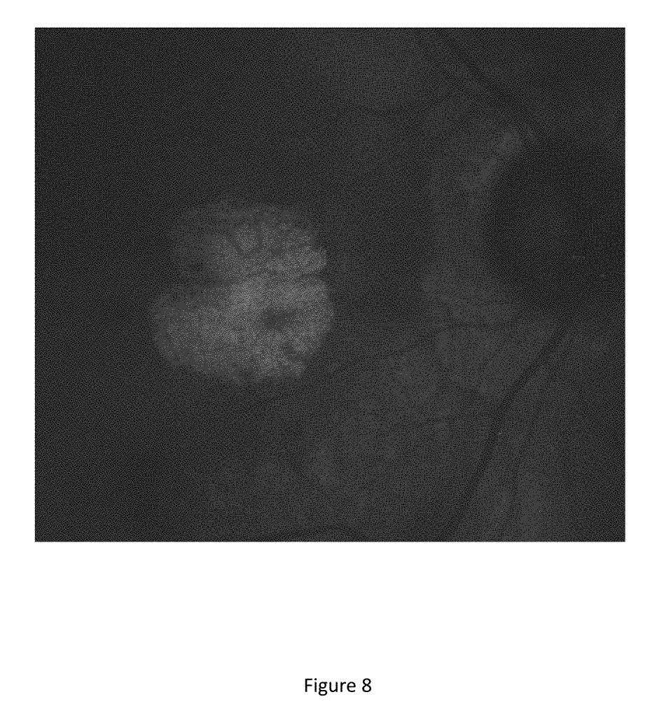

FIG. 8 is an example of the wide FOV, relatively low resolution, coarse-scale images that are used in the imaging apparatus and method of eye tracking. FIG. 8 is a single frame of an image from a WFSLO from the eye of a patient with cone-rod dystrophy. Individual live retinal images from a WFSLO typically contain a high percentage of low-contrast and dark regions, even if the optical system has been optimized. In a real-time image-based eye tracking system, where the tracking algorithm retrieves motion signals from real-time images to control one or more tracking mirrors, these low-contrast images can introduce artifacts or noise into the tracking signals. In the WFSLO image in FIG. 8, the resonant (fast) scanner scanned in the horizontal direction and the linear (slow) scanner scanned in the vertical direction. All notations between width and height are switched when the scanning direction is switched horizontally and vertically.

To obtain high-fidelity eye motion, the imaging apparatus and method tracks only blood vessels, and avoids the optic nerve disc because the optic disc is too rich in features. In general, a cross-correlation based tracking algorithm will fail when the optic nerve disc appears only on the reference image or only on the target image, but not when it appears in both images. Accordingly, the efficiency of the imaging apparatus and method is improved by not tracking the optic nerve disc.

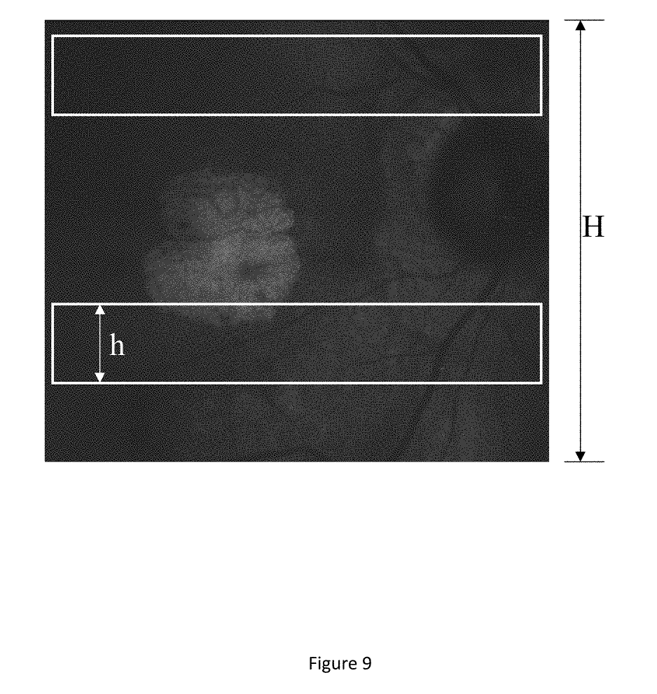

To achieve faster and smoother control for the tracking mirror, the field of view in the direction of slow scanning will be reduced to the height of the rectangle at faster frame rate, and the width of the image stays the same. For example, referring to FIG. 9, if the full image with height H has a frame rate f, and a smaller subset image with height h has frame rate F, these four parameters will satisfy the approximate equation,

F×H=f×h. (3)

The smaller image with height h that is captured at a high frame rate can be cropped from anywhere of the central part of the large, slow frame rate image, as long as the boundary of h does not run outside of H and the small image does not contain the optic nerve disc. The height h can be as small as possible, as long as the light power is under the ANSI safety level, and the small image contains enough features of blood vessels for cross-correlation. The height h can be set to no larger than ½ of H so that the h less frequently runs out of the boundary of H with fixational eye motions.

In one embodiment of the image-based tracking system, the large image with height H is used as a reference image and the small image with height h is used as a target image. A 2-D smoothing filter (e.g., Gaussian), followed by a 2D edge-detecting filter (e.g., Sobel) can be applied, if necessary, on both the reference image and the target image to retrieve the features of the blood vessels. A threshold can be applied on the filtered images to remove the artifacts caused by filtering, random noises, and/or a low-contrast background.

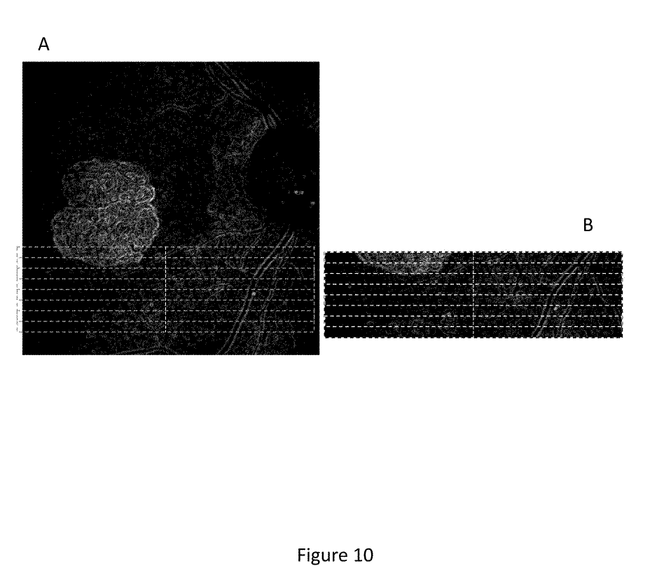

The method of image registration and eye-tracking involves cross-correlation between the reference and target images. As shown in FIG. 10, the reference image (10A) and the filtered target image (10B) are further divided into multiple strips with approximately the same width as the target image in FIG. 9, but with heights smaller than the height h in FIG. 9. Each strip in FIGS. 10A and 10B are further divided into two equally-sized sub-strips, i.e., one at the left and the other at the right, to aid in detecting eye torsion, which occurs frequently due to rotation of the eye or the head position. Cross-correlation can then be applied by comparing two corresponding strips, one from the reference image and one from the target image.

In an integrated eye tracking system, where the tracking mirror controlled by the SLO images can be used to dynamically steer the beam on another imaging system, such as an AOSLO or an OCT, relatively smooth motion from the tracking mirror is highly important. In one embodiment, smooth motion and control of the tracking mirror can be achieved as follows. The wide FOV SLO images are line-interleaved to achieve a doubled frame rate. With a doubled frame rate, the number of strips created per second in FIG. 10 is also doubled, and the update rate of the tracking mirror is doubled as well. A sub-pixel cross-correlation algorithm can be implemented to calculate eye motions from the SLO images. The optical resolution of a single pixel from the SLO system is usually on the order of tens of microns. A whole pixel of SLO motion applied on the tracking mirror will cause severe jitters on the AOSLO images, similar to microsaccades from human eyes. In one embodiment of the imaging apparatus and method, a digital low-pass filter can be applied on the motion traces to reduce unexpected spikes on the motion signals. In one embodiment, a high-resolution digital-to-analog converter (DAC) can be implemented to convert the (low-pass filtered) motion trace which is applied on the tracking mirror. In one embodiment, an analog low-pass filter can then be implemented after digital-to-analog conversion instead of, or in addition to, the digital low-pass filter.

Also described herein is a method for rapidly re-locking the tracking of a subject's eye position after a blink or some other type of interference with eye image tracking. Typically there are three statuses of fixational eye motion that must be considered during eye tracking: drift, blink, and microsaccade. Blinks can be discriminated by mean and standard deviation from individual image strips. When both mean and standard deviation of a strip drops below user-defined thresholds, this strip is treated as a blink frame, and the tracking mirror is suspended at its existing position. A microsaccade causes a single image strip to move several pixels in comparison to the previous strip. When multiple continuous strips move several pixels, the motion of the most recent strip is updated immediately on the tracking mirror. The number of multiple continuous strips required to cause an update on the tracking mirror can be determined by the user to balance tracking robustness and tracking accuracy. The update on tracking mirror is caused by a pulse signal to the tracking mirror to quickly adjust its status to compensate for a microsaccade. However, when only a single strip moves several pixels, it is not treated as a microsaccade strip, because this single motion is likely due to a miscalculation of the tracking algorithm as a result of minor variances or errors during cross-correlation between the target image strip and the reference image. In such a case, the position of the tracking mirror will be suspended at its current status. In motion associated with eye drift, the approach of using double frame rates and low-pass filters described above can be applied on the tracking mirror to control the tracking mirror smoothly.

In a multi-scale tracking system, e.g., the system shown in FIG. 4A, WFSLO tracking and AOSLO tracking are implemented in conjunction with each other as follows. The WFSLO continues eye tracking as long as the location of the fixation target does not change. When a region of interest (ROI) is determined on the WFSLO images, additional steering can be implemented to quickly steer AOSLO FOV to this area and zoomed in to get high-resolution live videos from the retina. Eye tracking, or optical stabilization, is started by using the AOSLO imaging in combination with AOSLO digital registration. When there is a rotation of the eye or the head, the reference frame of the WFSLO has to be adjusted. However, currently available systems have no hardware to optically rotate imaging FOVs of AOSLO and WFSLO, and the amount of rotation is beyond their capability for the detection of rotation and translation. If eye motion of the target frame m relative to the original reference frame is

(xₘ,yₘ,θₘ) (4)

and due to difficult eye/head rotation, this target frame m has to be updated as a new reference frame, then the future frame n will cross correlate with this frame m, with motion

(dxₙ,dyₙ,dθₙ) (5)

The net eye motion of frame n relative to the original reference is then

(xₘ+dxₙ,yₘ+dyₙ,θₘ+dθₙ) (6)

This approach enables the WFSLO to continuously track eye location, so that AOSLO imaging becomes efficient in steering its FOV to any ROI as along as it is in the steering range. At a particular fixation target, all reference frames are saved in an imaging session and their positions are determined by Equations (4)-(6). If the imaging session is stopped temporarily, i.e., the subject takes a break during the procedure, the AOSLO tracking system uses the most recent reference frame for the next imaging session. The location of AOSLO imaging FOV is passed to the WFSLO and recorded on a WFSLO image. Each AOSLO video has a unique WFSLO image to record its imaging position and size of FOV. The WFSLO notifies its tracking status to the AOSLO, e.g., microsaccade, blink, or tracking failure. In addition, the AOSLO notifies its status to the WFSLO, e.g., data recording and AOSLO tracking. Further, the WFSLO eye-tracking updates a new reference frame when the fixation target changes to a new location.

The imaging apparatus can use a number of different approaches to achieve smooth and robust control for the one or more tracking or steering mirrors (i.e., mirrors M2 and M3 in FIG. 4A). In the systems in both FIGS. 4A, 4B, and 5, a tracking algorithm is used to implement the control of M2 in the control loop of M1-WFSLO-M2. The control signals for M2 come from the real-time images of the WFSLO with cross-correlation technology. In the system of FIG. 4A, a second control loop, i.e., the closed control loop between AOSLO and M3 is also used in the image-based tracking method.

Referring again to FIG. 4A, light from the retina will be split into two channels via beam splitter M1, wherein one channel is sent to the wide FOV system (WFSLO), and the other channel to a tracking mirror M2. The light is then further directed to the second tracking mirror M3, and then related to the small FOV system (AOSLO). To reduce latency and increase accuracy on controlling the tracking mirror, the tracking mirror must be updated fast enough, e.g., every millisecond, to track eye motion. Accordingly, the imaging apparatus can also require a suitable electronics system for image processing.

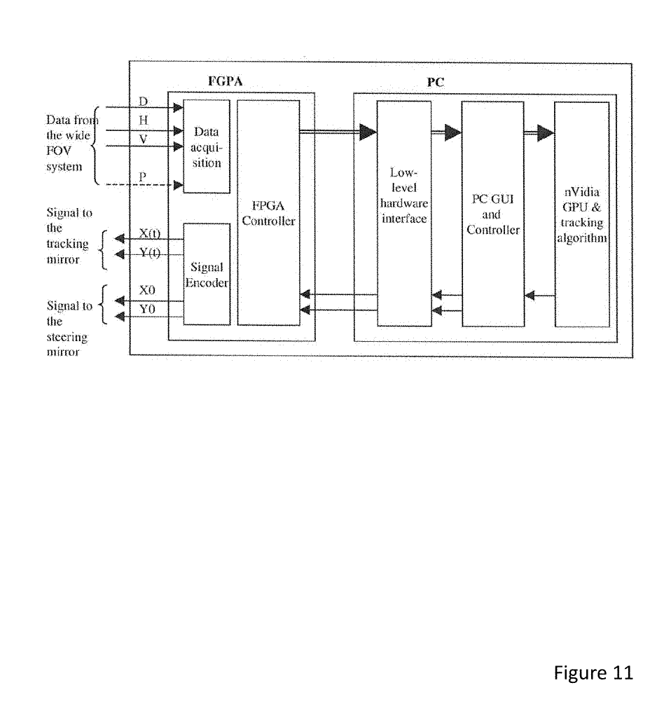

A schematic diagram of an exemplary embodiment of the electronics system for the wide FOV system is shown in FIG. 11, which is also implemented similarly for the closed-loop control of M3 in the AOSLO. In this system architecture, there are two modules: a FPGA module and a PC module. The FPGA module is responsible for real-time data acquisition from the optical system, flexible data buffering between FPGA and the host PC via a programmable controller, such as a PCle controller, and data encoding to one or multiple D/A converters to control external devices such as the tracking mirror and the steering mirror. Images from the wide FOV system can be in 1) analog format with analog data, H-sync, and V-sync, or 2) digital format with digital data, H-sync, V-sync, and pixel clocks. In analog format, an A/D converter is needed to digitize the images so that they can be sent to the FPGA. In digital format, FPGA can be programmed to sample parallel or serial digital data from the wide FOV optical system. In both cases, the digitized H-sync, V-sync and pixel clock can be used as common clocks throughout the entire FPGA application for buffering data from FPGA to PC through PCle interface. These three clocks are also used to synchronize D/A converters that output eye motion signals to the tracking mirrors. The FPGA are programmed to control any resolution of off-shelf A/D and D/A converters, from 8 bits to 16 bits or more.

The PC module is responsible for collecting images from the FPGA, sending the images to a graphics processing unit (GPU) for data processing, and then uploading eye motion signals and other control signals to the FPGA. The PC GUI and controller manage the hardware interface between the PC and the FPGA, the GPU eye-tracking algorithm, and the data flow between the FPGA, the PC CPU, and the GPU. In various embodiments, the GPU is a GPU manufactured by nVidia, or any other suitable GPU as would be understood by a person skilled in the art. In one embodiment, the FPGA is a Xilinx FPGA board (ML506 or ML605, Xilinx, San Jose). The selection of ML506 or ML 605 can depend on the format of images from the optical system, i.e., the ML506 can be used for analog data and the ML605 can be used for digital data. However, the FGPA can be any suitable board known in the art.

The architecture of the small FOV system can be similar to that of the wide FOV system described above, except that only one set of the steering mirror will be controlled, and the signals come from either WFSLO software or AOSLO software. However, in order to have maximum flexibility for additional functionality, the same Xilinx FPGA board (ML506 or ML605) used in the wide FOV system can be used in the small FOV system. This additional functionality can include, but is not limited to: real-time stabilized beam control to the retina, allowing for laser surgery with operation accuracy in sub-micrometers on the living retina; delivery of highly controllable image patterns with power modulation to the retina for scientific applications; and the real-time efficient montaging of retinal images.

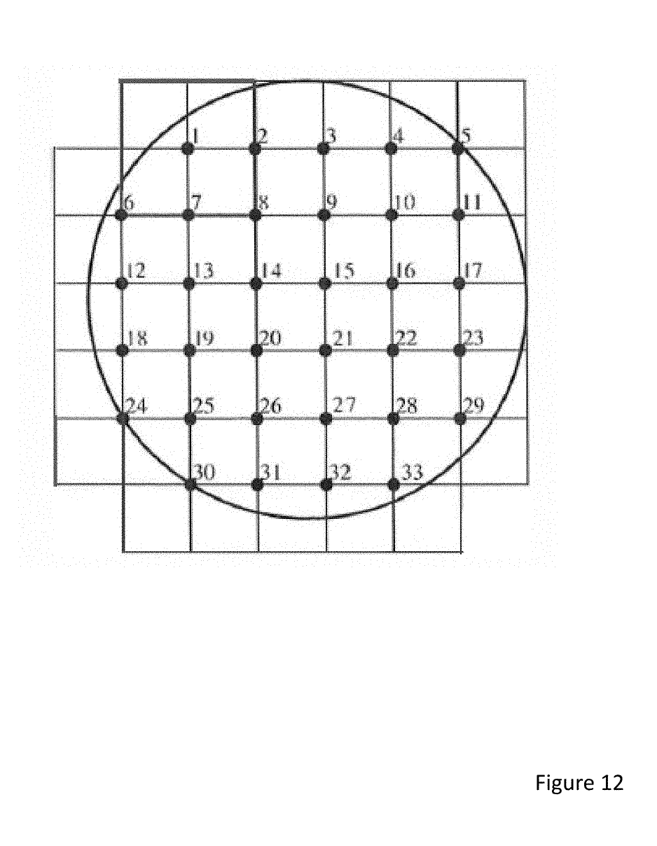

For example, FIG. 12 is a drawing representing the process of real-time retinal montaging. The circled area is the retina covered by the wide FOV system with low spatial resolution, and an area equivalent to four squares is covered by the small FOV system with high spatial resolution. To achieve a high-resolution image montage from the retina, the two systems can be programmed to direct the steering mirror to the locations of the dots with labels 1, 2, 3, etc., one at a time, wherein the four squares surrounding the targeted dot is covered by the small FOV system. In each location, the tracking mirror compensates for large eye motion, and the registration algorithm on the small FOV system removes the residual eye motions in real time, and then registers the images. In one embodiment, the software and hardware needs only about 5-10 seconds to register images in each location. The steering mirror can automatically be directed to the next location after the current one is finished. When the steering mirror sweeps through all predetermined locations (i.e., 33 in the example shown in FIG. 12), the software will automatically generate a large montage of the retina image. In such an embodiment, imaging of adjacent locations must be overlapped. The amount of overlapping required to maintain eye tracking depends on the residual eye motion on the small FOV system.

In one aspect, the imaging apparatus is an improvement over currently available technologies in that it can be used to process 512×512 pixel (or equivalent sized) warped images at 120 frames per second with high accuracy on a moderate GPU, for example an nVidia GTX560. The image tracking method takes advantage of the parallel processing features of GPUs, unlike currently available systems and methods that process less than 30 frames/second using a same or similar GPU.

The imaging apparatus and method can be used to perform the following: real-time image registration from a small and wide FOV SLO running at 30 frames/second or higher, e.g., in one embodiment, the frame rate can be 60 frames/second; real-time control of a tracking mirror to remove large eye motion on the small FOV SLO (1-2 degrees), by applying real-time eye motion signals from a large FOV SLO (10-30 degrees) every millisecond; and compensation for eye motion from an OCT in high accuracy with millisecond latency by applying real-time eye motion signals from a large FOV SLO (10-30 degrees) on the scanners of the OCT.

The method of image registration generally includes the following steps: 1) choose a reference frame, and divide it into several strips to account for image distortion; 2) retrieve a target frame, and also divide the target frame into the same number of strips as the reference frame; 3) perform cross-correlation between the reference strip and the target strip to calculate the motion of each target strip; and 4) register the target frame to the reference frame accounting for all motions of the target strips.

The speed and accuracy of the cross-correlation step, i.e., step 3, will determine the overall speed and accuracy of the image registration. Previous approaches to this step described in the prior art are not fast enough to enable image registration in real time. One reason for the lack of speed in these approaches is that they do not start the image registration algorithm until a whole frame is received by the host PC. This frame-level registration results in significant latency in controlling external devices such as scanners and/or tracking mirrors. For example, the shortest latency in such an approach is the frame rate of an imaging system, which can be about 33 milliseconds on a 30 frames/second system. Accordingly, when the computational latency from the GPU, CPU, and other processors are included, the total latency is generally significantly greater than 33 milliseconds.

The tracking method can be used to perform fast, real-time image registration by dramatically improving processing speed over currently known approaches. The tracking method is based on an algorithm that starts image registration as soon as a new strip from a target image is received by the host PC, instead of waiting for a whole frame to be delivered, as in current approaches. For example, a 520×544 image can be divided into 34 strips, each with a size of 520×16 pixels. Each strip is sent from the device to the host PC, which immediately sends it to the GPU where the motion of the strip is calculated.

On a testing benchmark with a nVidia GTX560 GPU, the computational time for processing each strip is about 0.17 millisecond. The dominant latency is from sampling the 520×16 strip which takes about 1.0 millisecond on a 30 frames/second system. Therefore, the total latency from input data to sending an output motion signal is about 1.5 milliseconds. In one embodiment, the sampling latency can be further reduced if the frame rate of an imaging system is increased.

In another aspect of the tracking method, the algorithm implemented in the GPU to achieve a computational time of 0.17 milliseconds per strip is also a significant improvement over the known art. Currently available methods mix parallel and serial processing on the GPU, resulting in busy data buffering between GPU and the host PC. To fully take advantage of the GPU computational capacity, the tracking method uses the GPU for parallel processing only, and converts all serial processing into parallel processing on the GPU. Further, the data communication between the GPU and the host PC is minimized. Specifically, to achieve optimal speed, raw image data is sent only once to the GPU. The GPU then performs all required processing in parallel, and returns only three parameters from the GPU to the host PC: the correlation coefficient and translations x and y. Further still, speed is improved by use the GPU shared memory and/or texture as much as possible, while avoiding the GPU global memory.

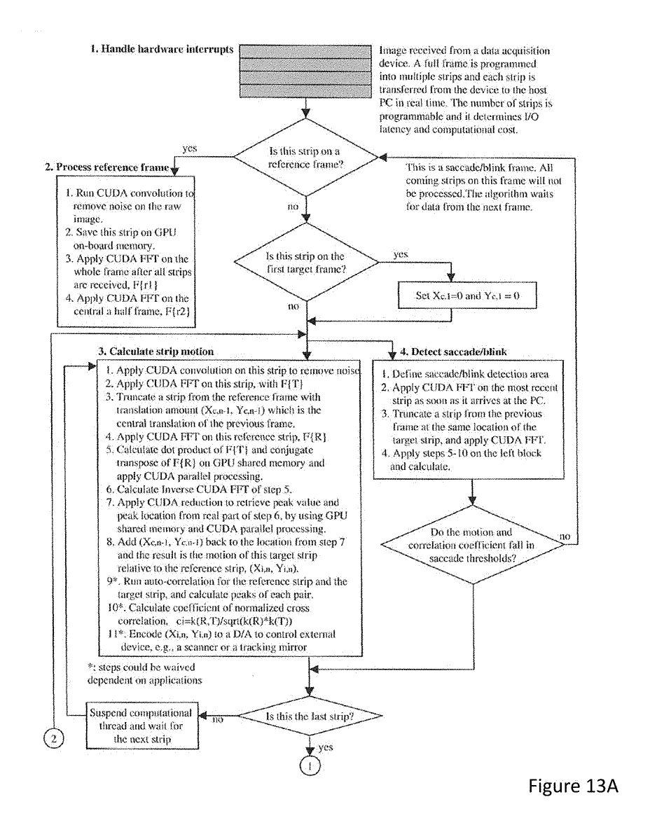

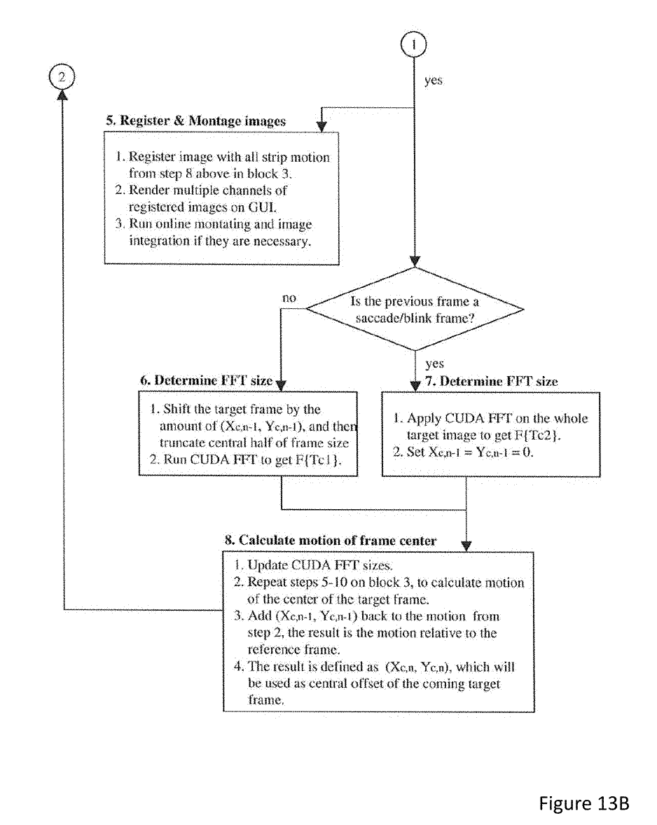

A flow chart of the algorithm for one embodiment of the tracking method is shown in FIGS. 13A and 13B. First, an image is acquired from a data acquisition device, e.g., an AOSLO or wide FOV SLO (step 510). The image, i.e., a single frame, is divided into multiple strips, and each strip is transferred from the device to the host PC in real time. In a preferred embodiment, as previously described herein, each strip is sent to the host PC immediately upon being generated instead of waiting for the entire frame to be generated and then divided into strips. The number of strips that the image is divided into is a programmable variable. The number of strips chosen can affect the I/O latency and computational cost.

If a strip is designated as coming from a reference frame (520) the strip will be processed using a reference frame protocol (525). Specifically, step 525 includes running a compute unified device architecture (CUDA) model implemented on the GPU, wherein noise is removed on the raw image, the strip saved on the GPU, and a CUDA fast Fourier transform (FFT) is applied to the whole frame or half frame. If a strip is not designated as coming from a reference frame, the strip is queried whether it is a strip on the first target frame (530). If the strip is on the first target frame, X_{c,1} and Y_{c,1} are each set to zero (535). If the strip is not on the first target frame, two protocols are run on the strip simultaneously. Specifically, a saccade/blink detection protocol is run (540) in conjunction with a protocol for calculating the strip motion (550). If a saccade or blink is detected (545), processing of all strips coming from this frame will be stopped and the algorithm will wait for the next frame (548). If a saccade or blink is not detected, the strip motion processing continues for the entire frame (550 & 555) until the last strip is received (560). After the last strip of a frame is received, the image is registered and, if necessary, montaged (570). Further, the FFT size is determined accordingly, based on whether the previous frame is a saccade/blink frame (580) or not a saccade/blink frame (575). The motion of the frame center is then calculated, which can be used to offset the next target frame as needed (585).

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. An ophthalmic laser surgery system, comprising:
an ophthalmic imaging apparatus,
a surgical light source, and
a steering mirror communicatively coupled with the imaging apparatus,
wherein the steering mirror is located in the pupil conjugate plain of a subject's eye, and
wherein when the steering mirror directs a laser beam from the surgical light source onto the subject's eye, backscattered light from the subject's eye is received by the imaging apparatus; the imaging apparatus tracks a motion of the subject's eye from the backscattered light; and the imaging apparatus sends a control signal based on the motion of the subject's eye to the steering mirror to direct the location of the laser beam.

2. The system of claim 1, further comprising a wavefront sensor for detecting an aberration in the subject's eye.

3. The system of claim 2, further comprising a beam splitter for splitting the beam of backscattered light, wherein a portion of the backscattered light is sent to the imaging apparatus and a portion of the backscattered light is sent to the wavefront sensor.

4. The system of claim 2, further comprising a stabilization/wavefront corrector communicatively coupled with the imaging apparatus and wavefront sensor.

5. The system of claim 4, wherein the stabilization/wavefront corrector sends a control signal to the steering mirror based on the motion and aberration of the subject's eye.

6. The system of claim 1, wherein the imaging apparatus is selected from the group consisting of: ocular coherence tomography (OCT) device, scanning laser ophthalmoscope (SLO), adaptive optics scanning light ophthalmoscope (AOSLO), fundus camera, line scan camera, pupil camera, or adaptive optics flood illumination camera.

7. The system of claim 1, wherein the surgical light source is a continuous wave (CW) laser, a pulsed laser, or a superluminescent diode (SLD).

8. The system of claim 1, further comprising a laser modulator.

9. The system of claim 1, wherein the laser modulator is selected from the group consisting of: direct laser diode modulator, mechano-optical isolator; acousto-optic modulator; electro-optic modulator; magneto-optical modulator; and optical isolator.

10. An ophthalmic laser surgery system, comprising:
an ophthalmic imaging apparatus,
a surgical light source,
a surgical steering mirror communicatively coupled with the imaging apparatus,
an imaging light source, and
an imaging steering mirror communicatively coupled with the imaging apparatus,
wherein the surgical steering mirror and imaging steering mirror are located in the pupil conjugate plain of a subject's eye, and
wherein when the imaging steering mirror directs a laser beam from the imaging light source onto the subject's eye, backscattered light from the subject's eye is received by the imaging apparatus; the imaging apparatus tracks a motion of the subject's eye from the backscattered light; and the imaging apparatus sends a control signal based on the motion of the subject's eye to the imaging steering mirror to direct the location of the imaging laser beam, and to the surgical steering mirror to direct the location of a surgical laser beam from the surgical light source.

11. The system of claim 10, further comprising a wavefront sensor for detecting an aberration in the subject's eye.

12. The system of claim 11, further comprising a dichroic mirror for directing a portion of the backscattered light to the wavefront sensor.

13. The system of claim 11, further comprising a stabilization/wavefront corrector communicatively coupled with the imaging apparatus and wavefront sensor.

14. The system of claim 13, wherein the stabilization/wavefront corrector sends a control signal to the imaging steering mirror and surgical steering mirror based on the motion and aberration of the subject's eye.

15. The system of claim 10, wherein the imaging apparatus is selected from the group consisting of: ocular coherence tomography (OCT) device, scanning laser ophthalmoscope (SLO), adaptive optics scanning light ophthalmoscope (AOSLO), fundus camera, line scan camera, pupil camera, and adaptive optics flood illumination camera.

16. The system of claim 10, wherein the surgical light source is a CW laser, a pulsed laser, or a SLD.

17. The system of claim 10, further comprising a laser modulator.

18. The system of claim 17, wherein the laser modulator is selected from the group consisting of: direct laser diode modulator, mechano-optical isolator; acousto-optic modulator; electro-optic modulator; magneto-optical modulator; and optical isolator.

19. A method for controlling the delivery of an ophthalmic laser, comprising:
providing an ophthalmic scan imaging apparatus and one or more ophthalmic light sources, wherein each light source is associated with a steering mirror, and the imaging apparatus is communicatively coupled to the one or more steering mirrors,
imaging a subject's eye with the imaging apparatus to detect one or more parameters of the subject's eye, and
adjusting the position of the one or more steering mirrors substantially simultaneously with the detection of the one or more parameters, thereby repositioning the delivery location on the subject's eye of the one or more light beams from the one or more light sources.

20. The method of claim 19, wherein the parameter is a motion of the subject's eye.

21. The method of claim 19, wherein the parameter is a feature on the subject's retina.

22. The method of claim 19, wherein at least one of the ophthalmic light sources is a surgical laser.

23. The method of claim 19, further comprising the step of modulating the one or more light beams based on the one or more parameters detected.

24. The method of claim 19, wherein the imaging apparatus comprises a wide field of view SLO and a small field of view apparatus.

25. The method of claim 24, wherein the direction of the wide field of view SLO fast-scanning axis is perpendicular to the small field of view apparatus fast-scanning axis, and the wide field of view SLO slow-scanning axis is perpendicular to the small field of view apparatus slow-scanning axis.
